(19) United States
(12) Patent Application Publication
(10) Pub. No.: US 2007/0166373 A1
(43) Pub. Date: Jul. 19, 2007

(54) Pre-fabricated semi-permeable capsule shells containing a semipermeable layer on a water-dispersible/soluble gelatinous shell for osmotic drug delivery system

(76) Inventor: Hasmukh Patel, Edison, NJ (US)

Correspondence Address: HASMUKH B. PATEL, 44 HANA ROAD, EDISON, NJ, 08817, US

(21) Appl. No.: 11/651,846

(22) Filed: Jan. 10, 2007

Related U.S. Application Data
(60) Provisional application No. 60/758,867, filed on Jan. 14, 2006.

(51) Int. Cl. A61K 9/64; A61K 9/24

(52) U.S. Cl. 424/456; 424/473

(57) ABSTRACT

The present invention is centered on the design, manufacturing and use of a novel capsule system made up of a bi- or multi-layered two piece capsule shell made of a water soluble/dispersible layer coated with water-insoluble semi-permeable membrane capsule shell. The therapeutic agent is formulated in the drug layer and the osmotic layer is compacted together. The osmotic layer is a rate controlling agent. This compacted core is then inserted in the body of the layered body followed by encapping with a layered cap (which is pre-drilled or drilled after inserting the core) followed by applying a sealing agent as a band where the body and the cap of capsule meet.

This application claims priority to the provisional application No. U.S. 60/758,867 with filing date of Jan. 14, 2006.

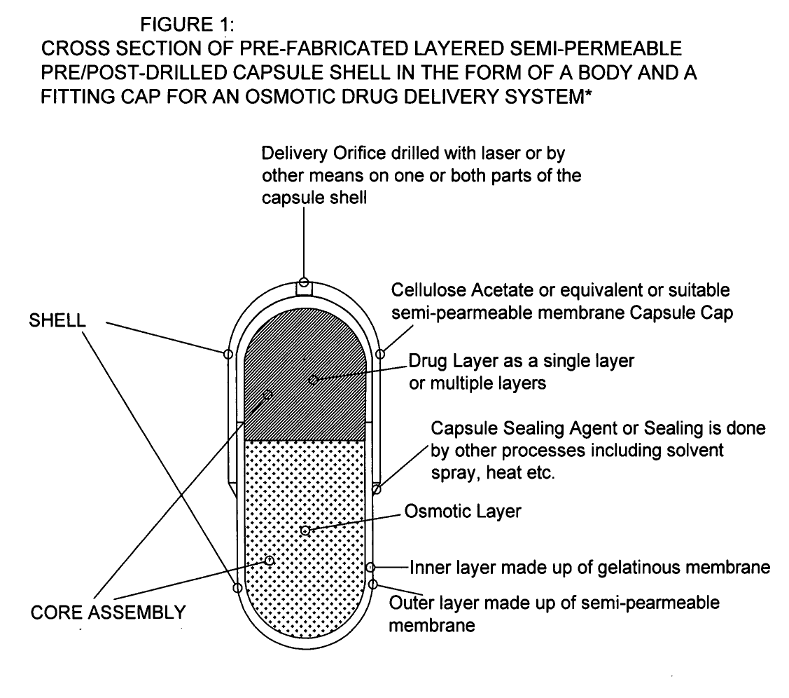

The present invention relates to pre-fabricated and pre- or post-drilled capsule shell made from a layered shell-wall with the outer membrane made of water insoluble, semi-permeable, polymer such as cellulose acetate or similar or equivalent suitable polymers or a mixture of the polymers (with additives such as plasticizers, coloring agent, opacifiers or other agents) and the inner wall of the shell made up of a gelatinous, water soluble, water dispersible, polymer such as gelatin, hydroxypropyl methylcellulose, microcrystalline cellulose or other water soluble/dispersible polymers for use in manufacturing of osmotic drug delivery systems. A typical shell is presented in Drawing FIG. 1 containing a tablet in which one part called drug layer contains a nutritional supplement or a therapeutic agent and the other part is osmotic layer in nature.

These capsule shells can be produced by a pre-fabrication process similar to the one used in producing conventional hard gelatin capsule shells. In this method pins designed for body or cap of the shell are first dipped in solution of natural, synthetic or semi-synthetic water soluble/dispersible polymers, including gelatin, hydroxypropyl methyl cellulose, microcrystalline cellulose or similar hydrophilic polymers or their mixture containing additives. The coated pins are dried, partially or completely, and then a second coat of a water insoluble, semi-permeable, polymer (such as cellulose acetate or similar water insoluble polymer) is applied and then the shell is dried and removed from the pin. Instead of dipping process a spray coating method can also be used to coat the inner shell. The composition of the layered shell can be designed to make an outer semi-permeable membrane from synthetic or semi-synthetic polymer including cellulose acetate or a mixture thereof containing additives such as color, plasticizer, opacifiers or others and the inner layer is made of a gelatinous water soluble polymer. Once the shell is produced it can be drilled or can be fitted with the core and then drilled to provide an outlet for the release of the therapeutic or nutritional agent in human or mammalian body.

The manufacturing process for this dosage form for controlled release formulation is distinctly different from the conventional dosage. Whereas the conventional dosage form requires spray-pan coating of the bi-layered cores in the current invention these bi-layered cored are inserted in the body of the proposed shell followed by capping with a cap followed by sealing with a sealing agent made from the same polymer or a mixture of similar polymer solution. In other words the proposed shell will obviate a need for spray pan coating making the manufacturing process (1) more efficient, (2) more reliable, (3) reproducible and can be done at a pharmaceutical manufacturing facility with very little infrastructure as compared to the conventional spray-pan coating process besides many other advantages.

The products developed using capsule shells described here can be used for therapeutic or nutritional purpose in which case such shell and tablet assembly will deliver drug or nutritional supplement over a long period of time, typically 4 to 24 hours but not limited to this time range.

### BACKGROUND OF THE INVENTION

Osmotically controlled release drug delivery systems are known in pharmaceutical art. One of these systems is made by spray coating (in a coating pan) a batch of tablet cores made up of an active layer and an osmotic layer. These systems are used for delivering a therapeutically useful agent over a period of time to a mammal. In present invention a novel dosage form is claimed.

### BRIEF SUMMARY OF THE INVENTION

The present invention is centered on the design, manufacturing and use of a novel capsule system made up of a bi- or multi-layered two piece capsule shell made of a water soluble/dispersible layer coated with water-insoluble semi-permeable membrane capsule shell. The therapeutic agent is formulated in the drug layer and the osmotic layer is compacted together. The osmotic layer is a rate controlling agent. This compacted core is then inserted in the body of the layered body followed by encapping with a layered cap (which is pre-drilled or drilled after inserting the core) followed by applying a sealing agent as a band where the body and the cap of capsule meet.

### DETAILED DESCRIPTION OF THE INVENTION

The present invention is of a novel capsule form which can be used for the manufacturing of osmotic oral drug delivery system. The invention involves prefabrication of bi- or multi-layered capsule body and cap. The inner layer of this shell is made up of water soluble or dispersible hydrophilic polymer including HPMC, gelatin, microcrystalline cellulose, carboxy methylcellulose (or its sodium salt) but not limited to these agents. The outer layer, which is applied by dipping process or by spraying is made up of a semi-permeable water insoluble polymer such as cellulose acetate but not limited to this agent. This shell is then used to encapsulate a core tablet made of multiple layers in which one is osmotic layer and others are drug or nutrient containing layers. Once the core tablet is encapsulated, the shell is sealed by a “banding” process in which a sealing agent is applied where the body and the cap of the capsule shell meet. The cross section of a typical novel capsule dosage form is shown in the attached drawing.

What is claimed is:

1. Bi- and multi-layered hard shells made of two parts a body and a telescopically fitting cap where the composition of the shell's outer layer is a water insoluble semi-permeable membrane and the inner layer is made up of a natural, synthetic or semi-synthetic, gelatinous water soluble or dispersible polymer as shown in Drawing FIG. 1 for osmotic drug delivery with core tablet made of an osmotic layer and one or more drug or nutrient layers.

2. A new design of an oral osmotic drug delivery system is claimed in which unlike conventional spray pan coating of the tablet, the tablet is put in a semi-permeable two piece shell made up of a body and a fitting cap, drilled and sealed, in claim 1, for controlling drug delivery and the delivery rate.

3. In claim 1 the thickness of each layer of the shell is 1 micrometer to 5 millimeter and that these shells are made encompassing all sizes available for the conventional hard gelatin capsules including US or European size 000, 00, 0, 1, 2, 3, 4, 5 known in the pharmaceutical art (but not limited to these sizes) and that the shell is pre-drilled or can be drilled post-assemblage with the core inside.

4. In claim 1 the outer layer of the shell is made from synthetic or semi-synthetic polymers including cellulose acetate or similar or equivalent polymers containing optionally a coloring agent, plasticizer, opacifier or other additives as the outer layer and an inner layer made up of gelatinous, water soluble, hydrophilic polymer(s) such as gelatin, hydroxypropyl methyl cellulose, carboxymethyl cellulose (or its sodium) salt or synthetic or semi-synthetic polymer containing optionally a coloring agent, plasticizer, opacifier or other additives.

5. In claim 1 the rate controlling shell is pre-fabricated in form of a capsule shell (in two parts, a body and a cap) which is drilled with a hole in pre-fabrication stage or post drilled after inserting the osmotic and drug layer assembly.

6. In claim 1 the osmotic layer is made of polymer such as polyethylene oxide or similar or equivalent with 1,000,000 to 15,000,000 molecular weight with other additives including osmotic agent(s) and the drug layer is made of 1000 to 500,000 molecular weigh polyethylene oxide or similar or equivalent polymer containing therapeutic agent from 1 micro gram to 1 gram and other ingredients.

7. In claim 1 the shape of the core is a tablet in which the body is cylindrical with the parabolic or hemispherical ends or other suitable shapes such as parabola or conical.

8. The method of use of the shell in claim 1 is for making oral osmotic drug delivery system in which a core described in claim 6 is inserted in the shell followed by sealing the shell with water insoluble-polymer solution, solvent, heat, adhesive or other means or a combination of these agents.

9. The dosage form claimed in claim 1 is for use of delivering therapeutic or nutritional agent to a mammal.
